(19) United States
(12) Patent Application Publication
(10) Pub. No.: US 2018/0092874 A1
(43) Pub. Date: Apr. 5, 2018

(54) IDENTIFICATION AND USE OF VERY LONG CHAIN DICARBOXYLIC ACIDS FOR DISEASE DIAGNOSIS, CHEMOPREVENTION, AND TREATMENT

(71) Applicant: Lincoln Memorial University, Harrogate, TN (US)

(72) Inventor: Paul L. Wood, Sacramento, CA (US)

(21) Appl. No.: 15/284,219

(22) Filed: Oct. 3, 2016

(51) Int. Cl. A61K 31/202; A61K 31/336; G01N 33/92

(52) U.S. Cl. CPC A61K 31/202; G01N 33/92; A61K 31/336

(57) ABSTRACT

A method for determining colorectal cancer risk includes obtaining a blood sample of the subject, isolating serum or EDTA plasma from the blood sample, analyzing the serum or EDTA plasma to determine plasma levels of very long chain dicarboxylic acid (VLCDCA 28:4), comparing the determined plasmas level of VLCDCA 28:4 of the subject with a predetermined range of plasma levels of VLCDCA 28:4 of diagnosed subjects having colorectal cancer, and determining a colorectal cancer risk exists when the determined plasma level of VLCDCA 28:4 is within the predetermined range of plasma levels of VLCDCA.

### CROSS-REFERENCE TO RELATED APPLICATIONS

Not Applicable

### STATEMENT REGARDING FEDERALLY-SPONSORED RESEARCH OR DEVELOPMENT

Not Applicable

### BACKGROUND OF THE INVENTION

### 1. Field of Invention

The present general inventive concept relates to biomarker compounds used in detection of diseases, and more specifically, to very long chain dicarboxylic acids (hereinafter “VLDCA” or “VLDCAs”) and methods of using VLDCAs as biomarkers for the detection, chemoprevention, and treatment of various diseases, including, but not limited to, colorectal cancer and kidney cancer. The identified VLCDAs are endogenous anti-inflammatory and anti-proliferative lipids specific to humans.

### 2. Description of the Related Art

Cancer is a type of disease in which abnormal cells begin to divide without control and which can potentially invade other tissues. Cancer cells may spread to various parts of a patient's body through the patient's blood and/or lymph system. There are many types of cancers, of which colorectal cancer has one of the highest mortality rates. However, although there currently exists several early detection screening programs, such as colonoscopy, which have proven effective at detecting colorectal cancer, many people are reluctant to undergo such procedures due to cost and perceived invasiveness. As a result, several minimally-invasive serum-based tests have been developed that identify people who are at a higher risk of developing certain types of cancers, including kidney and colorectal cancer.

One such test involves non-targeted lipidomics analysis of serum from patients who have been diagnosed with colorectal cancer or pancreatic cancer. The lipid extracts within the serum are monitored to determine whether a number of masses between 444 and 555 atomic mass units (amu) decrease over a period of time. However, since the lipids have yet to be synthesized as analytical standards, these lipids have been previously misassigned as vitamin E metabolites, and subsequently, as very-long chain hydroxylated polyunsaturated fatty acids, with 1 carboxy function, 2 to 6 double bonds, and 2 to 4 hydroxy substitutions. As a result, none of these conjectured lipid candidates have been synthesized as analytical standards to validate the structural assumptions and improve the reliability of clinical assays for these biomarkers.

In view of the above, what is desired is an accurate assignment and identification of metabolic markers which may be used as early stage risk indicators in a method for detecting and diagnosing certain types of cancer, including, but not limited to, kidney and colorectal cancer.

### BRIEF SUMMARY OF THE INVENTION

It has been found that a decrease in the prevalence of certain long-chain hydrocarbon biomarker masses is often a prelude to a cancer diagnosis. Therefore, screening for low levels of specific identified long-chain hydrocarbon biomarkers has potential as a useful tool for early identification of cancer risk and as an indicator for additional cancer testing. In particular, heightened cancer risk or incipient cancer (for example, colorectal cancer or pancreatic cancer) is correlated with decrements in the presence of very-long chain dicarboxylic acids (VLCDCAs) with between 28 and 30 carbon atoms, between 0 and 1 hydroxy groups, and between 1 and 4 double bonds as well as with VLCDCAs with between 32 and 36 carbon atoms, 1 or 2 hydroxy groups, and between 1 and 4 double bonds. One particular very-long chain dicarboxylic acid (VLCDCA) with 28 carbons and 4 double bonds has potential as a diagnostic marker and as a supplement to provide protection against cancer development. This VLCDCA (hereinafter identified as VLCDCA 28:4n6) has formula (I) but does not exclude other variants for localization of the double bonds:

HOOC—(CH₂)₄—CH═CH—CH₂—CH═CH—CH₂—CH═CH—CH₂—CH═CH—(CH₂)₁₁—COOH (I).

In various example embodiments, aspects and advantages of the present general inventive concept may be achieved by providing a method for validation of VLCDCA 28:4 as a dicarboxylic acid which may, in some embodiments, include sequential derivatization of 1 carboxylic group with [2H4]taurine and methylation of the second carboxylic group with trimethylsilyl diazomethane. Reactions may also be monitored by inclusion of the internal standard [2H28]VLCDCA 26:0. In one embodiment, for the sequential derivatization of the 2 carboxylic functional groups of VLCDCA 28:4, to 1 milliliter of dried plasma lipid extract are added 50 μL of 2-chloro-1-methypyrinium iodide (15.2 mg per 10 milliliters of acetonitrile and 16.4 μL of trimethylamine). The samples are heated at 30° C. with shaking for 15 minutes, followed by the addition of 50 μL of [2H4]taurine (5 mg in 900 μL of distilled water and 100 μL of acetonitrile). The samples are heated at 30° C. with shaking for another 2 hours before being dried by vacuum centrifugation. Next, 100 μL of 2-propanol and 20 μL of trimethylsilyl diazomethane (2 M in hexane) are added and the samples heated at 30° C. with shaking for 30 minutes. Next 20 μL of glacial acetic acid are added to consume any remaining trimethylsilyl diazomethane. The samples are then dried by vacuum centrifugation prior to dissolution in a mixture of isopropanol, methanol, and chloroform (4:2:1) containing 15 mM ammonium acetate. The mixture is analyzed in negative ESI (140,000 resolution) to monitor the anion of the derivatized lipids. This involves the addition of 111.02931 ([2H4]taurine) and 14.01565 (trimethylsilyl diazomethane) amu yielding a product of 571.3845 (446.33960+111.02931+14.01565) and an anion of 570.3772 which is monitored with 0.53 ppm mass error (FIG. 2B). Similarly, the internal standard [2H28]VLCDCA 26:0 is sequentially reacted with [2H4]taurine and trimethylsilyl diazomethane to yield a product of 439.4351 (314.39016+111.02931+14.01565) and an anion of 438.4278 which is monitored with 0.46 ppm mass error.

In various example embodiments, aspects and advantages of the present general inventive concept may be achieved by providing a method for diagnosing a subjects risk for having colorectal cancer which includes obtaining a blood sample of the subject, isolating serum or EDTA plasma from the blood sample, analyzing the serum or EDTA plasma to determine plasma levels of very long chain dicarboxylic acid (VLCDCA 28:4), comparing the determined plasmas levels of VLCDCA 28:4 of the subject with a predetermined range of plasma levels of VLCDCA 28:4 of diagnosed subjects having colorectal cancer, and determining the subject's risk of having colorectal cancer when the determined plasma levels of VLCDCA 28:4 within the blood sample is within the predetermined range of plasma levels of VLCDCA 28:4.

In some example embodiments, the foregoing and/or other aspects and advantages of the present general inventive concept may be achieved by providing a method for diagnosing a subjects risk for having colorectal cancer, the method encompassing obtaining a blood sample of the subject; isolating serum or EDTA plasma from the blood sample; analyzing the serum or EDTA plasma to determine plasma levels of VLCDCA 28:4; comparing the determined plasmas levels of VLCDCA 28:4 of the subject with a predetermined range of plasma levels of VLCDCA 28:4 of diagnosed subjects having colorectal cancer; and determining the subject has colorectal cancer when the determined plasma levels of VLCDCA 28:4 within the blood sample is within the predetermined range of plasma levels of VLCDCA 28:4.

In some example embodiments, the foregoing and/or other aspects and advantages of the present general inventive concept may be achieved by providing a method of treating a subject having colorectal cancer, the method including administering to the subject a sufficient amount to treat colorectal cancer a very-long chain dicarboxylic acid.

In some embodiments, the very-long chain dicarboxylic acid includes a straight chain group that is a C28-36 aliphatic group.

In some embodiments, the very-long chain dicarboxylic acid includes a straight chain group with between one and four double bonds.

In some embodiments, the very-long chain dicarboxylic acid includes epoxide or hydroxy functional groups.

In some embodiments, the very-long chain dicarboxylic acid is a compound (VLCFA 28:4) of formula (I):

HOOC—(CH₂)₄—CH═CH—CH₂—CH═CH—CH₂—CH═CH—CH₂—CH═CH—(CH₂)₁₁—COOH (I).

In some example embodiments, the foregoing and/or other aspects and advantages of the present general inventive concept may be achieved by providing a method of validating a dicarboxylic acid 28:4 structure, the method encompassing obtaining a blood sample of a subject; isolating serum or EDTA plasma from the blood sample; storing the serum or EDTA plasma in a low temperature environment; mixing about 1 milliliter (mL) of methanol comprising 1 nanomole of [²H₂₈] dicarboxylic acid 16:0 to a sample containing about 100 microliters of serum or EDTA plasma; mixing about 1 mL of distilled water and about 2 ml of tert-butyl methylether with the sample; separating an organic layer from the sample; drying the upper organic layer; dissoluting the dried upper organic layer in a mixture of isopropanol, methanol, and chloroform and ammonium acetate; performing mass spectrometry on the dissolution; and quantiating anions of dicarboxylic acid using negative ion electrospray ionization.

In some embodiments, the blood sample of the subject is obtained by venipuncture.

In some embodiments, the low temperature environment includes a refrigerator and a freezer.

In some embodiments, the organic layer is separated from the sample using centrifugal force of about 3000 times gravity.

In some embodiments, the mixture of isopropanol, methanol, and chloroform is at a ratio of 4:2:1.

In some embodiments, the mixture includes about 15 millimolar (mM) of the ammonium acetate.

In some embodiments, the mass spectrometry is performed via direct infusion.

In some example embodiments, the foregoing and/or other aspects and advantages of the present general inventive concept may be achieved by supplying a method of providing a chemopreventive agent to a subject having low circulating levels of VLCDAs, the method including: administering to the subject a sufficient amount to act as a chemopreventive agent a compound of formula (I), a prodrug of (I), or an analog of (I):

HOOC—(CH₂)₄—CH═CH—CH₂—CH═CH—CH₂—CH═CH—CH₂—CH═CH—(CH₂)₁₁—COOH (I).

In other example embodiments of the present general inventive concept, the foregoing and/or other aspects and advantages of the present general inventive concept may be achieved by providing a method of validating a dicarboxylic acid 28:4 structure which includes obtaining a blood sample of a subject, isolating serum or EDTA plasma from the blood sample, storing the serum or EDTA plasma in a low temperature environment, mixing about 1 milliliter (mL) of methanol comprising 1 nanomole of [²H₂₈] dicarboxylic acid 16:0 to a sample containing about 100 microliters of serum or EDTA plasma, mixing about 1 mL of distilled water and about 2 ml of tert-butyl methylether with the sample, separating an organic layer from the sample, drying the upper organic layer, dissoluting the dried upper organic layer in a mixture of isopropanol, methanol, and chloroform and ammonium acetate, performing mass spectrometry on the dissolution; and quantiating anions of dicarboxylic acid using negative ion electrospray ionization.

The blood sample of the subject may be obtained by venipuncture. The low temperature environment may include a refrigerator and/or a freezer.

The organic layer may be separated from the sample by using a centrifugal force of about 3000 times gravity.

The mixture of isopropanol, methanol, and chloroform may be at a ratio of 4:2:1. The mixture may include about 15 millimolar (mM) of ammonium acetate. The mass spectrometry may be performed via direct infusion.

VLCDCA 28:4 is present in all human biofluids examined (plasma, synovial fluid, pleural fluid, cerebrospinal fluid, and umbilical cord plasma). VLCDCA 28:4 was not detectable in the plasma of dogs, cows, horses, or the non-human primates cynonologous or rhesus macaque. In contrast, VLCDCA 28:4 levels were detected in the plasma of chimpanzees, the closest living human relative of the non-human primates.

Additional aspects and advantages of the present general inventive concept will be set forth in part in the description which follows, and, in part, will be obvious from the description, or may be learned by practice of the present general inventive concept.

Other features and aspects may be apparent from the following detailed description, the drawings, and the claims.

### BRIEF DESCRIPTION OF THE SEVERAL VIEWS OF THE DRAWINGS

The following example embodiments are representative of example techniques and structures designed to carry out the objects of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings in which:

FIGS. 1A and 1B are tables illustrating a listing of VLCDCAs extracted from human blood plasma. The parent masses and masses of the derivatized (carboxy and hydroxyl functional groups) molecules are listed;

FIG. 2A presents the molecular anion of the parent molecule VLCDCA 28:4 having a spectrum molecular anion of 445.332 amu; (1.94 ppm mass error) from control plasma;

FIG. 2B is a graph validating the dicarboxylic structure of VLCDCA 28:4 having a molecular anion of 570.3772 amu (0.53 ppm mass error) by sequential derivatization of 1 carboxylic group with [²H₄]taurine and methylation of the second carboxylic group with trimethylsilyl diazomethane with control plasma extracts;

FIG. 2C is a graph validating the dicarboxylic structure of the stable isotope internal standard [²H₂₈]VLCDCA 26:0 which is sequentially reacted with [²H₄]taurine and trimethylsilyl diazomethane to yield an anion of 438.4278 amu which is monitored with 0.46 ppm mass error;

FIG. 3A is a table of VLCDCA levels in the plasma of different animal species and in different human biofluids;

FIG. 3B is a chart illustrating decreased VLCDCA 28:4 plasma levels in plasma of patients diagnosed with kidney cancer and colorectal cancer;

FIG. 4 is a table listing the human biofluid levels of VLCDCA 28:6 and assessment of levels in the plasma of other species; and

FIG. 5 is a table illustrating a listing of carboxylic ester prodrugs of dicarboxylic acids and corresponding structures.

### DETAILED DESCRIPTION OF THE INVENTION

Reference will now be made to the example embodiments of the present general inventive concept, examples of which are illustrated in the accompanying drawings and illustrations. The example embodiments are described herein in order to explain the present general inventive concept by referring to the figures. The following detailed description is provided to assist the reader in gaining a comprehensive understanding of the structures and fabrication techniques described herein. Accordingly, various changes, modification, and equivalents of the structures and fabrication techniques described herein will be suggested to those of ordinary skill in the art. The progression of fabrication operations described are merely examples, however, and the sequence type of operations is not limited to that set forth herein and may be changed as is known in the art, with the exception of operations necessarily occurring in a certain order. Also, description of well-known functions and constructions may be simplified and/or omitted for increased clarity and conciseness.

Numerous variations, modifications, and additional embodiments are possible, and accordingly, all such variations, modifications, and embodiments are to be regarded as being within the spirit and scope of the present general inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

It is noted that the simplified diagrams and drawings included in the present application do not illustrate all the various connections and assemblies of the various components, however, those skilled in the art will understand how to implement such connections and assemblies, based on the illustrated components, figures, and descriptions provided herein. Numerous variations, modification, and additional embodiments are possible, and, accordingly, all such variations, modifications, and embodiments are to be regarded as being within the spirit and scope of the present general inventive concept.

It has been found that a decrease in the prevalence of certain long-chain hydrocarbon biomarker masses is often a prelude to a cancer diagnosis. Therefore, screening for low levels of specific identified long-chain hydrocarbon biomarkers has potential as a useful tool for early identification of cancer risk and as an indicator for additional cancer testing. In particular, heightened cancer risk or incipient cancer (for example, colorectal cancer or pancreatic cancer) is correlated with decrements in very-long chain dicarboxylic acids (VLCDCAs) with between 28 and 30 carbon atoms, with between 0 and 1 hydroxy groups, and between 1 and 4 double bonds as well as with VLCDCAs with between 32 and 36 carbon atoms, with 1 or 2 hydroxy groups, and between 1 and 4 double bonds.

One particular very-long chain dicarboxylic acid (VLCDCA) with 28 carbons and 4 double bonds has potential as a diagnostic marker and as a supplement to provide protection against cancer development. This VLCDCA (hereinafter identified as VLCDCA 28:4n6) has formula (I):

HOOC—(CH₂)₄—CH═CH—CH₂—CH═CH—CH₂—CH═CH—CH₂—CH═CH—(CH₂)₁₁—COOH (I).

According to the present general inventive concept, in various embodiments, lipid extracts within human plasma or serum which have monitored decreases in a number of molecules having atomic masses between 444 and 555 amu in patients diagnosed with pancreatic or colorectal cancer are identified as VLCDCAs. With regard to a molecular anion having an atomic mass of 445.3323 amu., this lipid is identified, for the first time, as VLCDCA 28:4. According to certain example embodiments of the present inventive concept, conversion of VLCFAs to dicarboxylic acids first involve ω-oxidation of the fatty acid by microsomal CYP4F, followed by conversion to an aldehyde via alcohol dehydrogenase, and the final conversion to a VLCDCA by CYP4F or by fatty aldehyde dehydrogenase. The present inventive concept provides a characterization of VLCDCAs of up to 36 carbons in length.

According to various example embodiments of the present inventive concept, VLCDCAs up to 36 carbons in length may be used as lipid biomarkers of various cancers, such as for example colorectal, ovarian, prostate, and pancreatic cancers. The present general inventive concept provides an accurate identification of the VLCDCA biomarker masses between 444 and 555 amu., which have been monitored to decrease in number within lipid extracts of human plasma or serum from patients diagnosed with colorectal cancer and pancreatic cancer. Pursuant to the present inventive concept, these lipid biomarkers are identified as VLCDCAs with 1 to 4 double bonds and 0, 1, or 2 hydroxy substitutions.

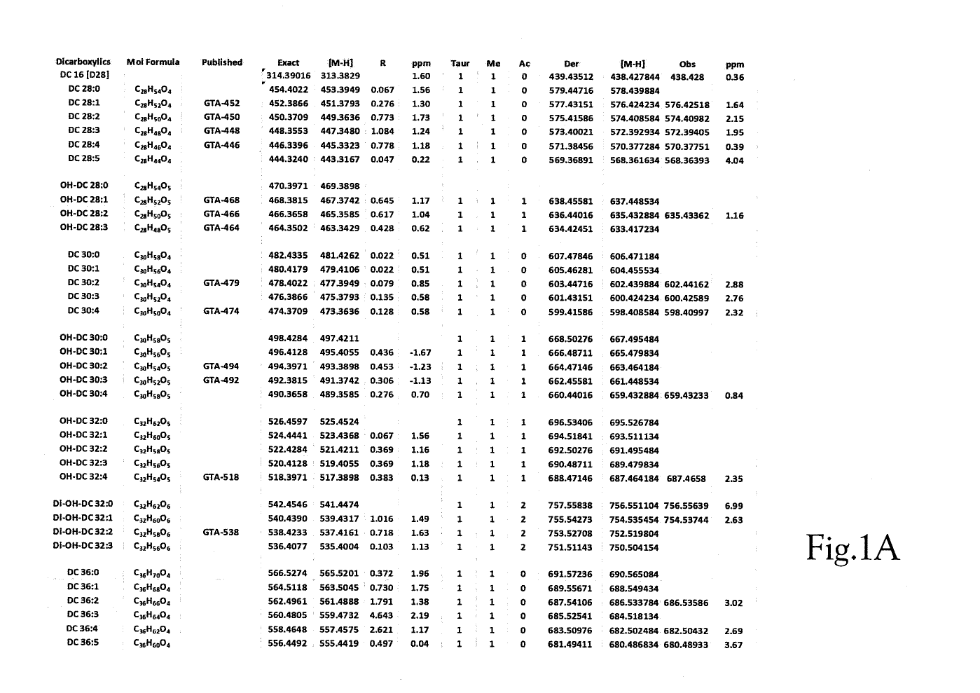

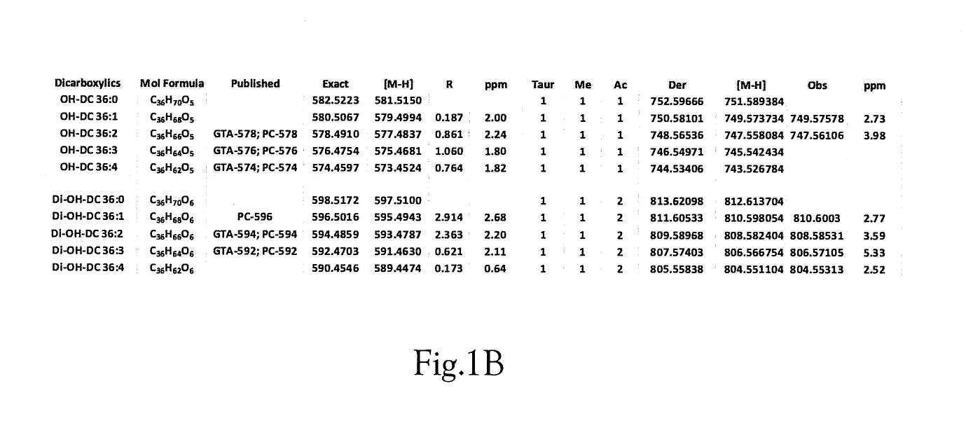

FIGS. 1A and 1B are tables illustrating a listing of VLCDCAs extracted from human blood plasma. Referring to FIGS. 1A and 1B, sequential fatty acid elongation involves elongation of very-long-chain fatty acids—4 (ELOVL4), an enzyme found in moderate levels in brain, spleen, pancreas, kidney, ileum, and lymph nodes, and in high levels in primate retina, thymus, epidermis, and germ cells. These very-long-chain fatty acids perform structural functions as fatty acid components of sphingomyelins and photophatidylcholines, serve in signal transduction roles, and are potential precursors to dicarboxylic acids.

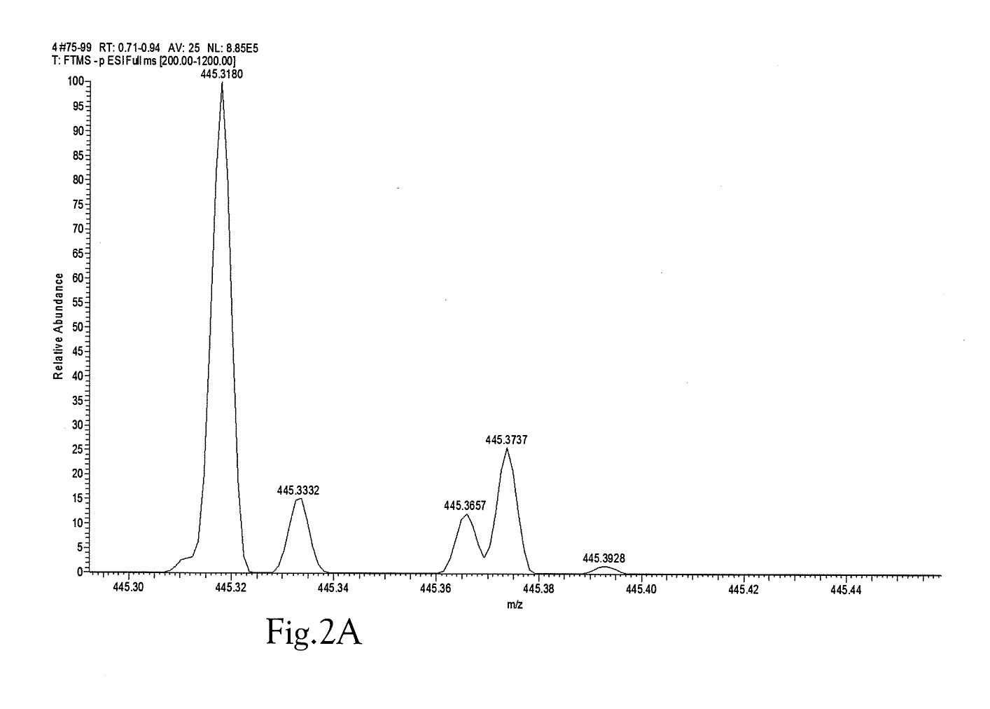

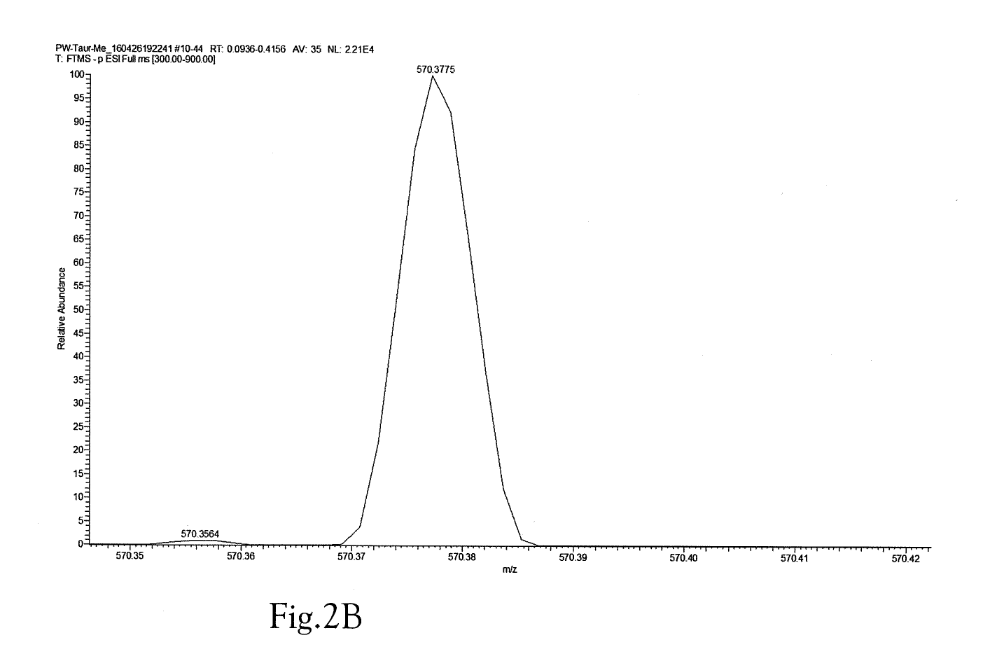

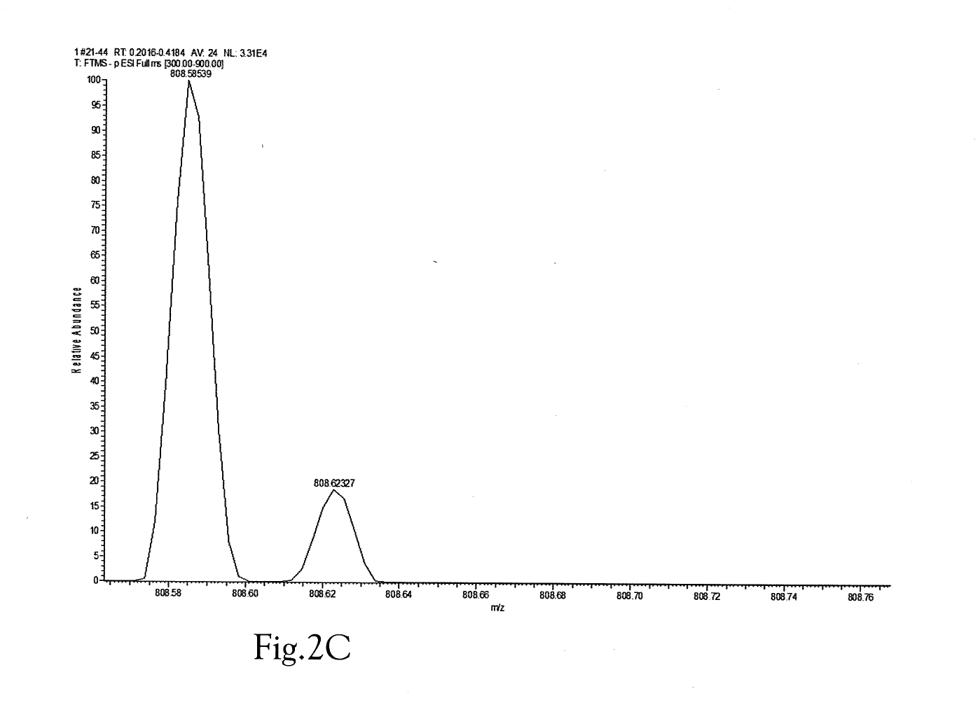

FIG. 2A is a graph of VLCDCA 28:4 having a spectrum molecular anion of 445.332 amu (1.94 ppm mass error) prior to sequential derivatization of 1 carboxylic group with [²H₄]taurine and methylation of the second carboxylic group with trimethylsilyl diazomethane. FIG. 2B is a graph validating a dicarboxylic structure (VLCDCA 28:4) having a molecular anion of 570.3772 amu (0.53 ppm mass error) by sequentially reacting organic extracts of control plasma with [²H₄]taurine and trimethylsilyl diazomethane. Referring to FIGS. 2A and 2B, a reaction of a lipid extract of 1000 uL of control plasma and [²H₄]taurine and trimethylsilyl diazomethane derivatizes both carboxylic acid groups. According to the present general inventive concept, the molecular anion 445.3323 amu is identified as a VLCDCA with 4 double bonds and no hydroxy substitutions. This lipid is properly identified and assigned as dicarboxylic acid 28:4, rather than the previous assignment as a fatty acid with 5 double bonds and 2 hydroxy substitutions (GTA-446).

According to one example embodiment of the present general inventive concept, a method of validating the dicarboxylic acid 28:4 structure is disclosed. In one embodiment, the method includes derivatization of the two carboxylic groups in VLCFA 28:4 by using [²H₄]taurine and trimethylsilyl diazomethane. This validation method includes obtaining blood samples collected by venipuncture and then isolating a sample of either serum or ethylenediaminetetraacetic acid (EDTA) plasma from the blood samples. The sample of serum and/or the EDTA plasma may, in certain embodiments, be stored in a low temperature environment (e.g. a refrigerator) or frozen to limit degradation of the sample prior to analysis.

According to certain example embodiments, a sample of approximately 100 microliters of serum and/or EDTA plasma is mixed with 1 milliliter (mL) of methanol containing 1 nanomole of [²H₂₈] dicarboxylic acid 16:0, of the type supplied, for example, by CDN Isotopes, 88 Ave. Leacota, PointeClaire, QC, H9R 1H1, to form a sample mixture. Next, 1 milliliter of distilled water and 2 milliliters of tert-butyl methylether are added to the sample mixture. The sample mixture is then agitated in an organic solvent in order to extract the lipid fraction. For example, in one embodiment, the sample mixture is shaken at a high speed (e.g., setting 9 of the Fisher Multitube Vortex) for approximately 30 minutes at room temperature. The sample mixture is then settled in order to separate an organic upper layer from the remainder of the sample. For example, in one embodiment, the sample mixture is transferred to a test tube and centrifuged at approximately 3000 times gravity at room temperature for approximately 10 minutes.

In one embodiment, upon the above-discussed settling of the sample mixture, approximately 1 milliliter of the upper organic layer is transferred to a 1.5 milliliter microtube and dried, for example by centrifugal vacuum evaporation, prior to dissolution of the dried upper organic layer portion in a mixture of isopropanol, methanol, and chloroform, at a ratio of 4:2:1, respectively, containing about 15 millimolar (mM) ammonium acetate. High resolution (e.g., 140,000 at 200 atomic mass unit) data acquisition, with sub-millimass accuracy, is then performed on the samples via direct infusion with an orbitrap mass spectrometer, for example, of the type manufactured and sold by Thermo Scientific under the trademark “Q Exactive™”. However, the present general inventive concept is not limited to a particular type or model of mass spectrometer. In embodiments in which multiple samples are processed according to the method invention simultaneously, in order to minimize ghost effects from one sample to the next, the input lines to the orbitrap mass spectrometer may be washed using methanol and a mixture of hexane and ethyl acetate, in a ratio of 3:2, respectively, between samples. Then, in negative ion electrospray ionization, the anions of dicarboxylic acid are quantitated, and from the acquired high-resolution dataset, the data may be reduced to provide a listing of VLCDCA, as illustrated in FIG. 1.

Validation of two carboxylic groups in VLCFA 28:4 was obtained by sequential derivatization of 1 carboxylic group with [²H₄]taurine and methylation of the second carboxylic group with trimethylsilyl diazomethane. The validation method includes adding approximately 1 milliliter of dried lipid extracts to 50 μL of 2-chloro-1-methypyrinium iodide (15.2 mg per 10 milliliters of acetonitrile and 16.4 μL of trimethylamine). The samples are heated at 30° C. with shaking for 15 minutes, followed by the addition of 50 μL of [²H₄]taurine (5 mg in 900 μL of distilled water and 100 μL of acetonitrile). The samples are heated at 30° C. with shaking for another 2 hours before being dried by vacuum centrifugation. Next, 100 μL of 2-propanol and 20 μL of trimethylsilyl diazomethane (2 M in hexane) are added and the samples heated at 30° C. with shaking for 30 minutes. Next 20 μL of glacial acetic acid are added to consume any remaining trimethylsilyl diazomethane. The samples are then dried by vacuum centrifugation. The mixture is then subjected to dissolution in a mixture of isopropanol, methanol, and chloroform, in ratios of 4:2:1, respectively, containing approximately 15 mM of ammonium acetate.

The mixture is analyzed in negative ESI (140,000 resolution) to monitor the anion of the derivatized lipids. This involves the addition of 111.02931 ([²H₄]taurine) and 14.01565 (trimethylsilyl diazomethane) amu yielding a product of 571.3845 (446.33960+111.02931+14.01565) and an anion of 570.3772 which is monitored with 0.53 ppm mass error (FIG. 2B). Similarly, the internal standard [²H₂₈]VLCDCA 26:0 is sequentially reacted with [²H₄]taurine and trimethylsilyl diazomethane to yield a product of 439.4351 (314.39016+111.02931+14.01565) and an anion of 438.4278 which is monitored with 0.46 ppm mass error. It will be recognized that the various quantities of materials used in the above-discussed embodiment of the method invention may vary, such that the method is performed using approximate ratios of materials according to the above-described embodiment. Such alternate embodiments according to the present general inventive concept, are contemplated herein, and should not be understood to depart from the present general inventive concept. Additionally, it is contemplated that the method invention may be used to simultaneously verify multiple samples at once, and such that, for example, multiple samples may be processed as described above without departing from the spirit and scope of the present general inventive concept.

In the case of dicarboxylic acids containing hydroxy functional groups, in various embodiments, the lipids first undergo sequential derivatization of 1 carboxylic group with [²H₄]taurine and methylation of the second carboxylic group with trimethylsilyl diazomethane. Next, the hydroxyl groups are derivatized with [²H₆]acetic anhydride. Specifically, the 2 carboxylic acid functions are derivatized as described above. The samples are then dried and 75 μL of pyridine and 75 μL [²H₆]acetic anhydride added. The samples are heated at 60° C., with shaking, for 1 hour and dried by vacuum centrifugation prior to dissolution in a mixture of isopropanol, methanol, and chloroform (4:2:1) containing 15 mM ammonium acetate. In the case of dihydroxy VLCDCA 36:2 (See FIG. 1B; GTA 594; PC 594), this yields a product of 809.5896 (594.48594+111.02931+14.01565+2*45.02939) which produces an anion of 808.5824 monitored with 3.68 ppm mass error (FIG. 2C). A complete list of the masses for endogenous VLCDCAs and their derivatives is presented in FIGS. 1A and 1B.

In alternative example embodiments, the data may be reduced simply as a ratio of a peak area of an endogenous lipid to a peak area of a stable isotope internal standard. However, the present general inventive concept is not limited thereto. That is, in alternative example embodiments, standard curves may be constructed for absolute quantitation, when analytical standards are available. In further example embodiments, VLCFAs may be quantitated by tandem mass spectrometry (MS²) or various other mass spectrometers, including, but not limited to, unit resolution mass spectrometry with a triple quadrupole instrument. However, the present general inventive concept is not limited thereto. In yet further example embodiments, various conventional chromatographic methods such as liquid chromatography, capillary zone electrophoresis, and supercritical fluid chromatography may be used as alternatives to direct infusion. However, the present general inventive concept is not limited thereto.

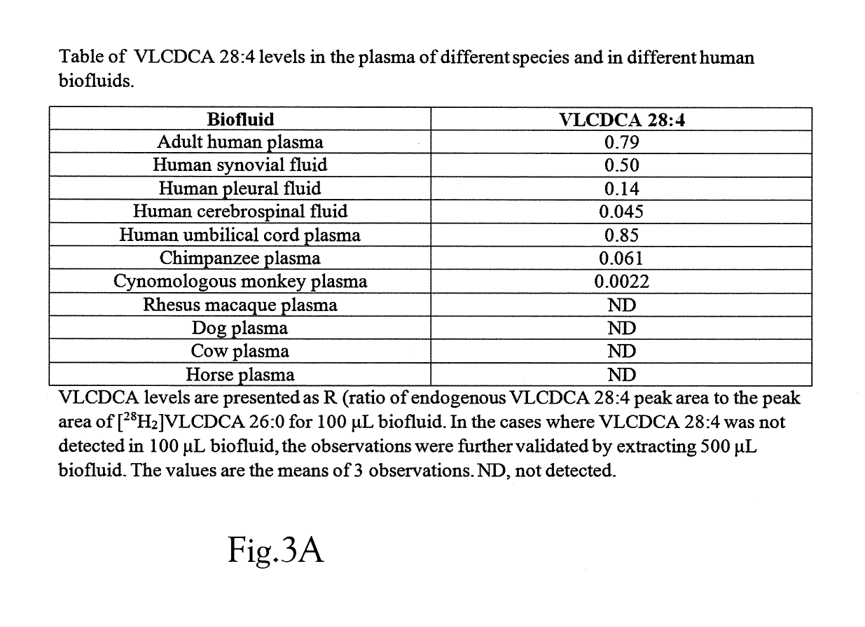

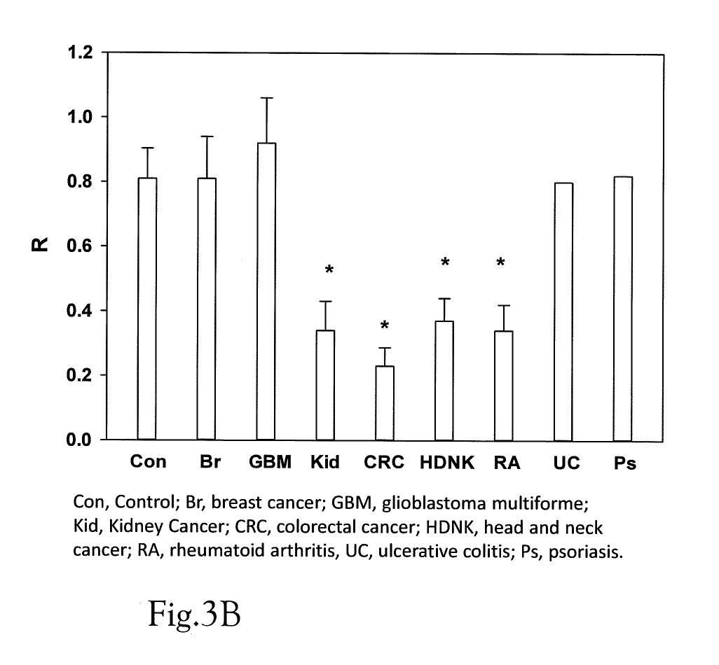

FIG. 3A is a table of VLCDCA levels in the plasma of different animal species and in different human biofluids. These data show that VLCDCA is only present in the blood of higher primates indicating that this lipid represents a late evolutionary development. In humans, VLCDCA is present in a wide diversity of biofluids in addition to blood plasma.

FIG. 3B is a chart illustrating decreased VLCDCA 28:4 plasma levels in plasma of patients with kidney cancer, colorectal cancer, head and neck cancer, and rheumatoid arthritis. Referring to FIG. 3B, the plasma levels of VLCDCA 28:4 are decreased in plasma of patients with colorectal cancer, as compared to plasma levels of VLCDCA 28:4 within a control group. In addition, the plasma levels of VLCDCA 28:4 are also decreased in plasma of patients with kidney cancer, head and neck cancer, and rheumatoid arthritis. It will be recognized that these examples are illustrative of the viability for use of VLCDCA 28:4 plasma levels as an indicator of various types of cancer, and the present general inventive concept is not limited to these cancer types. In addition, plasma levels of VLCDCA 28:4 are biomarkers of chronic and progressive inflammatory diseases like rheumatoid arthritis but not restricted to rheumatoid arthritis.

Clinically, decrements in these biomarker masses (masses between 444 and 555) have been detected prior to cancer development. In addition, these biomarker masses are not restored post-surgery to remove identified cancerous tissues, which suggests that these biomarker masses are not derived from the cancerous tissues and may represent intrinsic chemoprotective factors. In another example embodiment of the present general inventive concept, supplements of these factors, including VLCDCAs such as VLCDCA 28:4, may be provided to people who have been identified as having a higher risk of developing certain types of cancers to provide protection against cancer development. In certain instances, purified fractions of these identified lipids from human plasma have been observed to possess both anti-inflammatory and anti-proliferative properties.

According to an example embodiment of the present inventive concept, the identified lipid biomarker VLCDCA 28:4 is generated by a conversion of VLCFAs. This conversion first involves w-oxidation of the VLCFA 28:4 (VLCFA 28:4n6) by microsomal CYP4F, followed by conversion to an aldehyde via alcohol dehydrogenase, and the final conversion to VLCDCA 28:4n6 by CYP4F or by fatty aldehyde dehydrogenase. While VLCDCAs of up to 26 carbons have been previously reported, the present inventive concept provides a characterization of VLCDCAs of up to 36 carbons in length. However, the present general inventive concept is not limited thereto.

According to other features of the present general inventive concept, methods of quantitation of serum or plasma levels of the identified lipid biomarker VLCDCA 28:4 within a subject may be used to monitor these lipids as risk factors for developing a plurality of cancers, including, but not limited to, colorectal, kidney, prostate, and pancreatic cancers. For example, in one embodiment, VLCFAs may be quantitated by MS2 on various other mass spectrometers including unit resolution mass spectrometry with a triple quadrupole instrument. In addition, chromatographic methods may also be used as alternatives to direct infusion methods, which may include liquid chromatography, capillary zone, electrophoresis, and supercritical fluid chromatography. However, the present general inventive concept is not limited thereto.

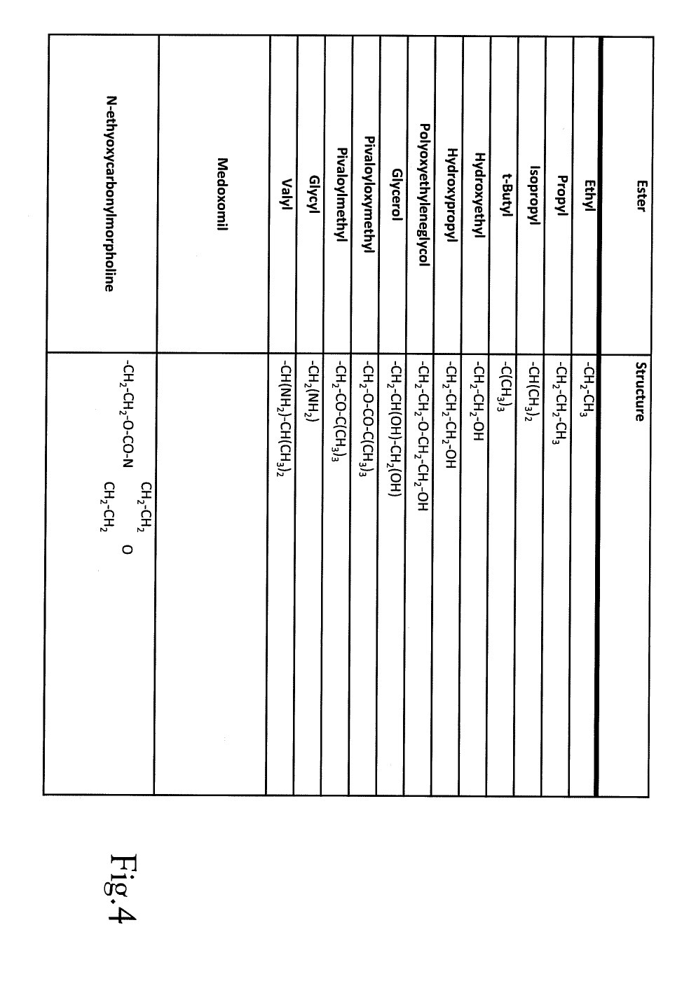

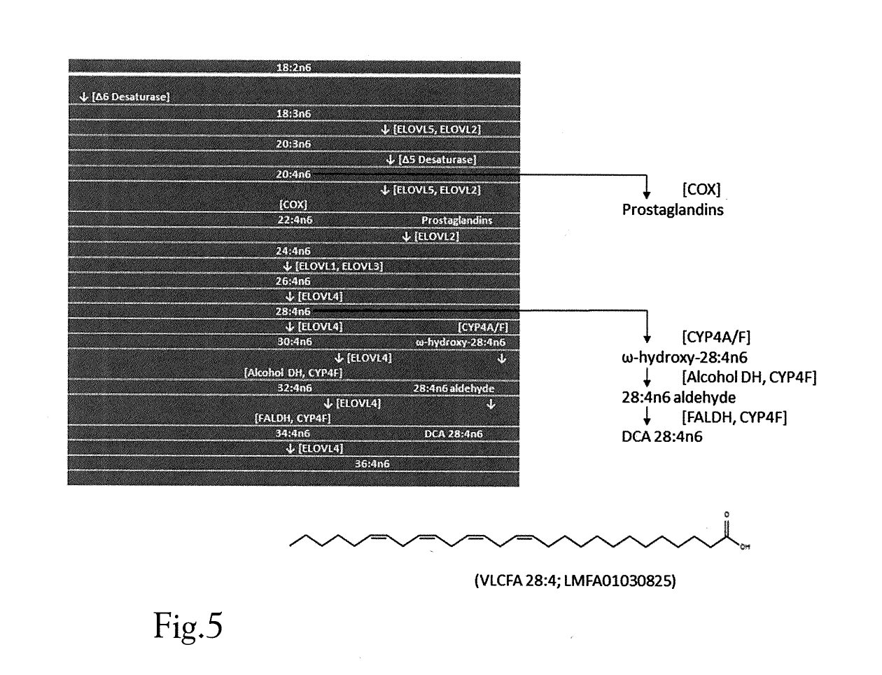

FIG. 4 is a table illustrating a listing of carboxylic ester prodrugs of dicarboxylic acids and corresponding structures. In addition, and in accordance with certain features of the present general inventive concept, the identified lipid biomarker VLCDCA 28:4 or potential esters of VLCDCA 28:4 may be used in the development of various pharmaceutical analogs or prodrugs of these lipids, which may be used as cancer treatment medication or as cancer chemoprevention medicines. Referring to FIG. 5, in various example embodiments, mono- and di-esters of the identified lipid biomarker VLCDCA 28:4 may be used in the development of prodrugs. In alternative example embodiments, the identified lipid biomarker VLCDCA 28:4 according to present general inventive concept may be provided in pharmaceutical compositions including a carrier or in combination with various other agents or drugs. In other example embodiments, the identified lipid biomarker VLCDCA 28:4 according to present general inventive concept may be provided as supplements, nutraceuticals, and/or combined with various other foods. In still other example embodiments, the identified lipid biomarker VLCDCA 28:4 according to present general inventive concept may be administered to a subject diagnosed with at least one of a plurality of cancers, including, but not limited to, colorectal, kidney, prostate, and pancreatic cancers, in an amount sufficient to treat, prevent, and/or mitigate the cancer.

### Method of Treating: Colorectal Cancer

In example embodiments, the present general inventive concept provides a method of treating a subject having colorectal cancer. In alternative example embodiments, the present general inventive concept also provides a chemopreventive agent and a method of treating a subject having low circulating levels of VLCDCAs with the chemopreventive agent. The treatment method includes administering to the subject having colorectal cancer or low circulating levels of VLCDCAs a sufficient amount of VLCDCAs to increase the level of VLCDCAs circulating in the blood a compound according to the formula (I), a prodrug of (I), or an analog of (I):

HOOC—(CH₂)₄—CH═CH—CH₂—CH═CH—CH₂—CH═CH—CH₂—CH═CH—(CH₂)₁₁—COOH (I)

However, the present general inventive concept is not limited thereto. That is, in other example embodiments, structural analogs of compound (I) and/or prodrug esters of compound (I) may also be developed to provide superior and/or improved bioavailability (BA).

### Method of Treating: Pancreatic Cancer

In other example embodiments, the present general inventive concept provides a method of treating a subject having pancreatic cancer and as a chemopreventive agent in individuals with low circulating levels of VLCDCAs. The treatment method includes administering to the subject having pancreatic cancer or low circulating levels of VLCDCAs a sufficient amount of VLCDCAs to increase the level of VLCDCAs circulating in the blood a compound according to the formula (I), a prodrug of (I), or an analog of (I):

HOOC—(CH₂)₄—CH═CH—CH₂—CH═CH—CH₂—CH═CH—CH₂—CH═CH—(CH₂)₁₁—COOH (I)

However, the present general inventive concept is not limited thereto. That is, in other example embodiments, structural analogs of compound (I) and/or prodrug esters of compound (I) may also be developed to provide superior and/or improved bioavailability (BA).

### Method of Treating: Prostate Cancer

In alternative example embodiments, the present general inventive concept provides a method of treating a subject having prostate cancer and as a chemopreventive agent in individuals with low circulating levels of VLCDCAs. The treatment method includes administering to the subject having prostate cancer or low circulating levels of VLCDCAs a sufficient amount of VLCDCAs to increase the level of VLCDCAs circulating in the blood a compound according to the formula (I), a prodrug of (I), or an analog of (I):

HOOC—(CH₂)₄—CH═CH—CH₂—CH═CH—CH₂—CH═CH—CH₂—CH═CH—(CH₂)₁₁—COOH (I)

However, the present general inventive concept is not limited thereto. That is, in other example embodiments, structural analogs of compound (I) and/or prodrug esters of compound (I) may also be developed to provide superior and/or improved bioavailability (BA).

Other VLCDCAs (listed in FIG. 1A), as well as structural analogs or prodrug esters of these VLCDCAs, are also potential therapeutic candidates for increasing the level of VLCDCAs circulating in the blood and treating colorectal cancer.

While the present general inventive concept has been illustrated by description of several example embodiments, and while the illustrative embodiments have been described in detail, it is not the intention of the applicant to restrict or in any way limit the scope of the general inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings. Additional modifications will readily appear to those skilled in the art. Accordingly, departures may be made from such details without departing from the spirit or scope of applicant's general inventive concept.

### REFERENCES

1. Ritchie S A, Ahiahonu P W, Jayasinghe D, Heath D, Liu J, Lu Y, Jin W, Kavianpour A, Yamazaki Y, Khan A M, Hossain M, Su-Myat K K, Wood P L, Krenitsky K, Takemasa I, Miyake M, Sekimoto M, Monden M, Matsubara H, Nomura F, Goodenowe D B. (2010) Reduced levels of hydroxylated, polyunsaturated ultra long-chain fatty acids in the serum of colorectal cancer patients: implications for early screening and detection. BMC Med. 8:13.
2. Ritchie S A, Heath D, Yamazaki Y, Grimmalt B, Kavianpour A, Krenitsky K, Elshoni H, Takemasa I, Miyake M, Sekimoto M, Monden M, Tomonaga T, Matsubara H, Sogawa K, Matsushita K, Nomura F, Goodenowe D B. (2010) Reduction of novel circulating long-chain fatty acids in colorectal cancer patients is independent of tumor burden and correlates with age. BMC Gastroenterol. 10:140.
3. Ritchie S A, Tonita J, Alvi R, Lehotay D, Elshoni H, Myat S, McHattie J, Goodenowe D B. (2013) Low-serum GTA-446 anti-inflammatory fatty acid levels as a new risk factor for colon cancer. Int J Cancer. 132:355-62.
4. Ritchie S A, Goodenowe D., Khan M A, Ahiahonu P W K. (2012) Hydroxy fatty acid compounds and uses thereof for disease treatment and diagnosis. EPA2459510 A1.
5. Goodenowe D., Ritchie S (2007) Methods for the diagnosis of colorectal cancer and ovarian cancer by the measurement of vitamin e-related metabolites. WO2007030928 A8.
6. Ritchie S A, Akita H, Takemasa I, Eguchi H, Pastural E, Nagano H, Monden M, Doki Y, Mori M, Jin W, Saj obi T T, Jayasinghe D, Chitou B, Yamazaki Y, White T, Goodenowe D B. (2013) Metabolic system alterations in pancreatic cancer patient serum: potential for early detection. BMC Cancer. 13:416.
7. Ritchie S A, Chitou B, Zheng Q, Jayasinghe D, Jin W, Mochizuki A, Goodenowe D B. (2015) Pancreatic cancer serum biomarker PC-594: Diagnostic performance and comparison to CA19-9. World J Gastroenterol. 21:6604-12.
8. Ritchie S A, Bingham E. (2008) Methods for the diagnosis of ovarian cancer health states and risk of ovarian cancer health states. PCT/CA2008/000270.
9. Ritchie S A, Bingham E. (2007) Biomarkers useful for diagnosing prostate cancer, and methods thereof. PCT/CA2007/000469.
10. Ritchie S A, Jayasinghe D, Davies G F, Ahiahonu P, Ma H, Goodenowe D B. (2011) Human serum-derived hydroxy long-chain fatty acids exhibit anti-inflammatory and anti-proliferative activity. J Exp Clin Cancer Res. 2011 30:59.
11. Higashi T, Ichikawa T, Inagaki S, Min J Z, Fukushima T, Toyo'oka T. (2010) Simple and practical derivatization procedure for enhanced detection of carboxylic acids in liquid chromatography-electrospray ionization-tandem mass spectrometry. J Pharm Biomed Anal. 52:809-18.
12. Björkhem I. (1978) On the quantitative importance of omega-oxidation of fatty acids. J Lipid Res. 1978 19:585-90.
13. Boddupalli S S, Estabrook R W, Peterson J A. (1990) Fatty acid monooxygenation by cytochrome P-450BM-3. J Biol Chem. 1990 265:4233-9.
14. Sanders R J, Ofman R, Dacremont G, Wanders R J, Kemp S. (2008) Characterization of the human omega-oxidation pathway for omega-hydroxy-very-long-chain fatty acids. FASEB J. 22:2064-71.
15. Wood P L, Unfried G, Whitehead W, Phillipps A, Wood J A (2015) Dysfunctional plasmalogen dynamics in the plasma and platelets of patients with schizophrenia. Schizophrenia Res 161:506-510.
16. Senanayake V K, Jin W, Mochizuki A, Chitou B, Goodenowe D B. (2015) Metabolic dysfunctions in multiple sclerosis: implications as to causation, early detection, and treatment, a case control study. BMC Neurol. 15:154.
17. Ruiz-Orera J, Hernandez-Rodriguez J, Chiva C, Sabidó E, Kondova I, Bontrop R, Marqués-Bonet T, Albà M M. (2015) Origins of De Novo Genes in Human and Chimpanzee. PLoS Genet. 11:e1005721.
18. Prado-Martinez J, Sudmant P H, Kidd J M, Li H, Kelley J L, Lorente-Galdos B, Veeramah K R, Woerner A E, O'Connor T D, Santpere G, Cagan A, Theunert C, Casals F, Laayouni H, Munch K, Hobolth A, Halager A E, Malig M, Hernandez-Rodriguez J, Hernando-Herraez I, Prüfer K, Pybus M, Johnstone L, Lachmann M, Alkan C, Twigg D, Petit N, Baker C, Hormozdiari F, Fernandez-Callejo M, Dabad M, Wilson M L, Stevison L, Camprubi C, Carvalho T, Ruiz-Herrera A, Vives L, Mele M, Abello T, Kondova I, Bontrop R E, Pusey A, Lankester F, Kiyang J A, Bergl R A, Lonsdorf E, Myers S, Ventura M, Gagneux P, Comas D, Siegismund H, Blanc J, Agueda-Calpena L, Gut M, Fulton L, Tishkoff S A, Mullikin J C, Wilson R K, Gut I G, Gonder M K, Ryder O A, Hahn B H, Navarro A, Akey J M, Bertranpetit J, Reich D, Mailund T, Schierup M H, Hvilsom C, Andres A M, Wall J D, Bustamante C D, Hammer M F, Eichler E E, Marques-Bonet T. (2013) Great ape genetic diversity and population history. Nature. 499:471-5.

Having thus described the aforementioned invention, what is claimed is:

1. A method for determining colorectal cancer risk, the method comprising:
isolating serum or EDTA plasma from a blood sample;
analyzing the serum or EDTA plasma to determine a plasma level of VLCDCA 28:4;
comparing the determined plasma level of VLCDCA 28:4 with a predetermined range of plasma levels of VLCDCA 28:4, the predetermined range of plasma levels of VLCDCA 28:4 predetermined from multiple subjects diagnosed as having colorectal cancer; and
determining a colorectal cancer risk exists when the determined plasma level of VLCDCA 28:4 is within the predetermined range of plasma levels of VLCDCA 28:4.

2. A method of treating a subject having colorectal cancer, the method comprising:
determining plasma levels of VLCDCA 28:4 in blood of a subject having colorectal cancer;
administering to the subject an amount of a very-long chain dicarboxylic acid sufficient to increase the plasma levels of VLCDCA 28:4 in the blood of the subject having colorectal cancer.

3. The method of claim 2, wherein the very-long chain dicarboxylic acid includes a straight chain group that is a C28-36 aliphatic group.

4. The method of claim 2, wherein the very-long chain dicarboxylic acid includes a straight chain group with between one and four double bonds.

5. The method of claim 2, wherein the very-long chain dicarboxylic acid includes an epoxide functional group.

6. The method of claim 2, wherein the very-long chain dicarboxylic acid is a compound of formula (I):
HOOC—(CH₂)₄—CH═CH—CH₂—CH═CH—CH₂—CH═CH—CH₂—CH═CH—(CH₂)₁₁—COOH (I).

7. A method of validating a dicarboxylic acid 28:4 structure, the method comprising:
isolating serum or EDTA plasma from a blood sample;
storing the serum or EDTA plasma in a low temperature environment;
mixing about 1 milliliter (mL) of methanol comprising 1 nanomole of [4H₂₈] dicarboxylic acid 16:0 with the isolated serum or EDTA plasma;
mixing about 1 mL of distilled water and about 2 ml of tert-butyl methylether with the isolated serum or EDTA plasma;
separating an organic layer from the isolated serum or EDTA plasma;
drying the organic layer;
dissoluting the dried organic layer in a mixture of isopropanol, methanol, chloroform, and ammonium acetate;
performing mass spectrometry on the dissolution; and
quantiating anions of dicarboxylic acid using negative ion electrospray ionization to validate the dicarboxylic acid 28:4 structure.

8. The method of claim 7, wherein the blood sample is obtained by venipuncture.

9. The method of claim 7, wherein the low temperature environment is provided by a refrigerator or a freezer.

10. The method of claim 7, wherein the organic layer is separated from the isolated serum or EDTA plasma using centrifugal force of about 3000 times gravity.

11. The method of claim 7, wherein the mixture of isopropanol, methanol, and chloroform is at a ratio of 4:2:1.

12. The method of claim 11, wherein the mixture includes about 15 millimolar (mM) of the ammonium acetate.

13. The method of claim 7, wherein the mass spectrometry is performed by a direct infusion methodology.

14. A method of providing VLCDAs to a subject having low circulating levels of VLCDAs, the method comprising:
determining the level of VLCDAs circulating in the blood of a subject;
administering to the subject a sufficient amount of VLCDAs to increase the level of VLCDAs circulating in the blood of the subject, the administered VLCDAs including a compound of formula (I):
HOOC—(CH₂)₄—CH═CH—CH₂—CH═CH—CH₂—CH═CH—CH₂—CH═CH—(CH₂)₁₁—COOH. (I).
